(19) United States
(12) Patent Application Publication
(10) Pub. No.: US 2020/0395113 A1
(43) Pub. Date: Dec. 17, 2020

(54) IMPLEMENTING AND VERIFYING COMPLIANCE WITH CURRENT STANDARDS OF CARE

(71) Applicant: Rodney Paul Lusk, Creede, CO (US)

(72) Inventor: Rodney Paul Lusk, Creede, CO (US)

(21) Appl. No.: 16/901,930

(22) Filed: Jun. 15, 2020

Related U.S. Application Data
(60) Provisional application No. 62/861,392, filed on Jun. 14, 2019.

(51) Int. Cl. G16H 20/00; G16H 40/20; G16H 10/60; G16H 80/00

(52) U.S. Cl. CPC G16H 20/00; G16H 80/00; G16H 10/60; G16H 40/20

(57) ABSTRACT

Recommendations of disease-specific clinical practice guidelines are represented as compliance rules that are used by an application that generates a user interface on a computing device used by a clinician during a patient encounter. The clinician enters values in fields in the user interface to implement the recommendations. An analysis of how closely the clinician has complied with the recommendations is automatically generated and added to a patient medical record. Rules that represent revised clinical practice guidelines are automatically distributed to clinician computing devices, thereby facilitating introduction of best practice for specific diseases, and substantially improving the treatment of those diseases.

### TECHNICAL FIELD

The subject matter of this disclosure is generally related to healthcare, and more particularly to implementation of and compliance with current disease-specific clinical practice guidelines.

### BACKGROUND

Each medical specialty has a professional organization such as an academy, society or commission that creates disease-specific clinical practice guidelines. Guidelines are most often presented as formal documents, but they may also be conveyed in less formal vehicles such as systematic reviews and MetaAnalysis. Most guidelines are created with input from multiple academies and stakeholders interested in a specific disease. Therefore, these clinical practice guidelines for a disease are widely accepted as the current best practices or “standard of care” in terms of performance measures, diagnostic criteria, treatment modalities, and outcomes assessment. Disease-specific Clinical Practice Guidelines (CPG) are meant to be comprehensive, so they tend to be lengthy and complex, e.g., 50-150 pages in printed form. Furthermore, clinical practice guidelines are periodically revised as best practices change. Unfortunately, the recommendations contained in the clinical practice guidelines can take years to be implemented within the specialty community. This is due in part to the massive amount of medical literature that is being published, and the little time clinicians can spend reading this literature. On average clinicians only spend approximately 5 hours a month reading the literature. Since the timely implementation of disease-specific, best practice recommendations does not happen, effective treatment of a disease within a specialty is substantially reduced.

### SUMMARY

All examples, aspects and features mentioned in this document can be combined in any technically possible way.

In accordance with some implementations a method for implementing and verifying compliance with current standards of care comprises: creating computer-readable Compliance Information that represents recommendations of current clinical practice guidelines associated with a specific disease, wherein the Compliance Information comprises compliance rules for evaluating adherence with the recommendations as a function of values of key elements; distributing the Compliance Information to each of a plurality of computing devices, each comprising a microprocessor and memory; contemporaneous with a patient encounter wherein the specific disease is indicated by a preliminary diagnosis, generating a patient-specific record with one of the computing devices by: generating a user interface on the computing device based on the distributed Compliance Information; prompting gathering of data needed to assign values to the key elements; evaluating the compliance rules using the values assigned to the key elements; prompting compliance with recommendations for which compliance is not indicated by evaluated ones of the compliance rules; and generating a verification of compliance with the current standards of care in response to compliance with the recommendations as indicated by evaluated ones of the compliance rules.

In accordance with an aspect a compliance automation system for implementing and verifying compliance with current standards of care comprises: at least one compute node that generates computer-readable Compliance Information that represents recommendations of current clinical practice guidelines associated with a specific disease, wherein the Compliance Information comprises compliance rules for evaluating adherence with the recommendations as a function of values of key elements; a non-volatile storage repository via which the Compliance Information is distributed to each of a plurality of clinician computing devices, each comprising a microprocessor and memory; and program code that runs on one of the clinician computing devices contemporaneous with a patient encounter for which the specific disease is indicated by a preliminary diagnosis, the program code comprising instructions that: generate a user interface on the clinician computing device based on the distributed Compliance Information; prompt gathering of data needed to assign values to the key elements; evaluate the compliance rules using the values assigned to the key elements; prompt compliance with recommendations for which compliance is not indicated by evaluated ones of the compliance rules; and generate a verification of compliance with the current standards of care in response to compliance with the recommendations as indicated by evaluated ones of the compliance rules.

A wide variety of other implementations, features and aspects will be apparent in view of the detailed description and figures.

### BRIEF DESCRIPTION OF THE FIGURES

FIG. 1 illustrates a method for implementing and verifying compliance with the recommendations in a current or revised clinical practice guideline.

FIG. 2 illustrates a system in which the method of FIG. 1 is implemented.

FIG. 3 illustrates a process for defining structured recommendations extracted from a practice guideline.

FIG. 4 illustrates a process for defining the KBI key elements associated with the Recommendation Definitions.

FIG. 5 illustrates implementation of Compliance Rules for recommendations associated with a specific disease.

FIG. 6 illustrates automatic creation of a disease-specific UI on the clinician computing device in a scenario in which an external program reads Compliance Information and modifies a general UI to cover a disease-specific patient encounter.

FIG. 7 illustrates automatic creation of a disease-specific UI on the clinician computing device in a scenario in which a standalone application or a Cloud-based application generates the disease-specific UI.

FIG. 8 illustrates automatic creation of a disease-specific UI on the clinician computing device in a scenario in which a Cloud-based subroutine is called by an external program.

FIG. 9 illustrates a table that identifies the disease to be structured, the textual Guideline title, and a link to the textual Guideline source.

FIG. 10 illustrates a template for a Recommendation Definition table that defines the structured components of a recommendations in a disease-specific CPG.

FIG. 11 illustrates a table of structured Recommendation Definitions derived from a CPG for pediatric tonsillitis.

FIG. 12 illustrates the second half of the Recommendation Definitions table for pediatric tonsillitis.

FIG. 13 illustrates a template for a KBI Definition table.

FIG. 14 illustrates, as an example, the first section of a KBI Definition table for pediatric tonsillitis.

FIG. 15 illustrates the next section of the KBI Definition table for pediatric tonsillitis.

FIG. 16 illustrates the next section of the KBI Definition table for pediatric tonsillitis.

FIG. 17 illustrates the next section of the KBI Definition table for pediatric tonsillitis.

FIG. 18 illustrates a template of a table used to define the machine-readable Compliance Rules for the set of Recommendations.

FIG. 19 illustrates the first section of a table of the Compliance Rules for recommendations for pediatric tonsillitis.

FIG. 20 illustrates the second section of a table of the Compliance Rules for recommendations for pediatric tonsillitis.

FIG. 21 illustrates machine readable object data structure of compiled Compliance Rules for pediatric tonsillitis.

FIG. 22 illustrates a table displaying the relationships among the recommendations for pediatric tonsillitis.

FIG. 23 illustrates a value list for the key elements for pediatric tonsillitis.

FIG. 24 illustrates Counselling summary for pediatric tonsillitis.

FIG. 25 also illustrates auxiliary information to support a Patient Education KBI.

FIG. 26 illustrates the first section of a generated Patient Encounter User Interface for pediatric tonsillitis.

FIG. 27 illustrates the second section of a generated Patient Encounter User Interface for pediatric tonsillitis.

FIG. 28 illustrates a runtime assessment of full compliance and incompatibilities for the values entered into the User Interface during a patient encounter where the preliminary diagnosis was pediatric tonsillitis.

FIG. 29 illustrates a generated face page for a patient encounter UI, where pediatric tonsillitis is chosen as the preliminary diagnosis for pediatric tonsillitis.

### DETAILED DESCRIPTION

Some aspects, features and implementations described herein may include machines such as computers, electronic components, optical components, and processes such as computer-implemented steps. It will be apparent to those of ordinary skill in the art that the computer-implemented steps may be stored as computer-executable instructions on a non-transitory computer-readable medium. Furthermore, it will be understood by those of ordinary skill in the art that the computer-executable instructions may be executed on a variety of tangible processor devices. For ease of exposition, not every step, device, or component that may be part of a computer or data storage system is described herein. Those of ordinary skill in the art will recognize such steps, devices, and components in view of the teachings of the present disclosure and the knowledge generally available to those of ordinary skill in the art. The corresponding machines and processes are therefore enabled and within the scope of the disclosure.

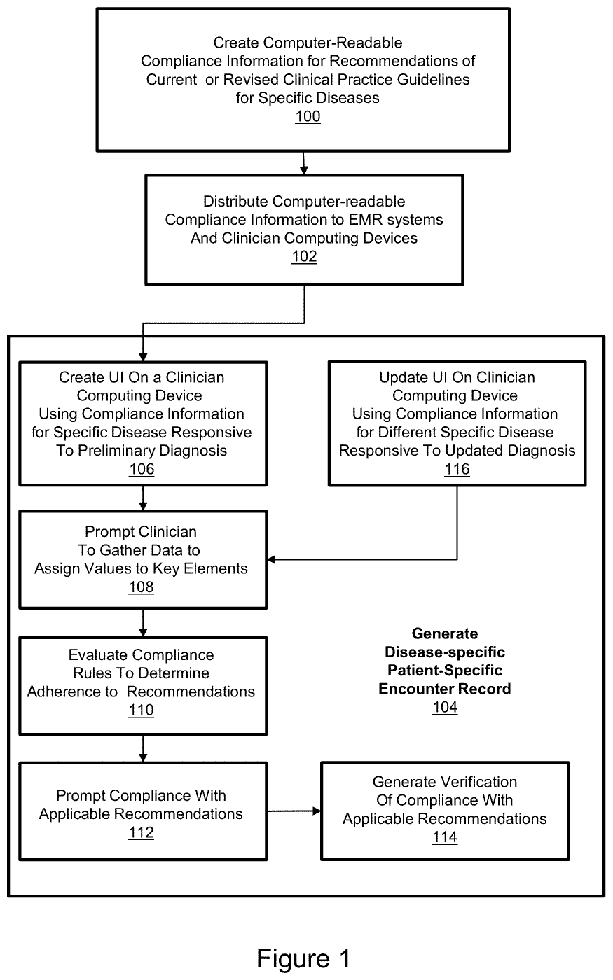

FIG. 1 illustrates a method for implementing and verifying compliance with the recommendations in a current or revised clinical practice guideline. Each clinical practice guideline (CPG) for each specific disease includes recommendations associated with treatment of that specific disease. Some CPGs use the term Action Statement as a more directed form of a Recommendation. The CPG may also indicate the importance of Recommendations or Action Statements that may be characterized by various levels such as Optional, Recommended, and Strongly-recommended. Computer-readable Compliance Information can be generated for the recommendations in a disease-specific, current, or revised CPG as indicated in step 100. In other words, Compliance Information can be generated and recorded for multiple specific diseases. The Compliance Information may include logic statements that enable the evaluation of compliance with a recommendation based on values entered for key elements in the logical statement. These key elements are referred to as Knowledge Base Items (KBIs). This approach may identify a minimal set of key elements that are required to assure compliance with a best practice recommendation. This advantageously improves efficiency as values of other elements described in the best practice document may not have to be supplied during a patient encounter to comply with the set of recommendations. The values of KBIs may indicate the presence or absence of a key element or quantify the key element. Examples of information that can be used to assign values to key elements include information commonly included in a chronology of a patient encounter. Some or all the Compliance Information is distributed to subscribing electronic medical record (EMR) systems and clinician computing devices as indicated in step 102. Some or all the Compliance Information for each specific disease is stored in a repository in the Cloud. The Compliance Information is used by the clinician computing devices, either directly or cooperatively with an EMR system, to generate a disease-specific, patient-specific encounter record as indicated in step 104. In response to a preliminary diagnosis, e.g., during a patient encounter, the Compliance Information for a specific disease indicated by the preliminary diagnosis is used to generate a disease-specific and patient-specific user interface (UI) on a clinician computing device as indicated in step 106. The UI prompts the clinician to gather data to assign values to the key elements required to evaluate the compliance rules associated with the specific disease as indicated in step 108. As the values are entered into the clinician computing device via the UI, compliance rules are evaluated to determine the adherence to recommendations associated with the CPG for the specific disease as indicated in step 110. Not all the recommendations are always applicable. For example, various recommendations may be determined to be inapplicable based on information gathered from the patient. The clinician computing device prompts the clinician to comply with all applicable recommendations as indicated in step 112. In some implementations the applicability is also a function of importance, e.g., optional recommendations may be considered inapplicable. Revision of a clinical practice guideline may result in modification, deletion, and addition of recommendations. Consequently, a clinician who is unfamiliar with the current written version of the clinical practice guideline for a specific disease will be made aware of values needed to adhere to the updated recommendations in step 112. A verification of compliance with applicable recommendations is generated as indicated in step 114. This may include verification of implementation of all applicable recommendations, an indication of which applicable recommendations were implemented, a score based on which applicable recommendations were implemented, and any combinations thereof. Thus, recommendations of a current or revised clinical practice guideline are implemented without a requirement that the clinician study a textual version of the clinical practice guideline. Further, a verification of compliance provides a useful addition to the patients' medical record.

In some instances, the clinician may update the preliminary diagnosis. As indicated in step 116, the UI on the clinician computing device is updated using compliance rules associated with a different disease indicated by an updated diagnosis. Steps 108, 110, 112 and 114 are then implemented using the compliance rules for that different disease. Data entered for the first diagnosis is retained and applied to the second diagnosis.

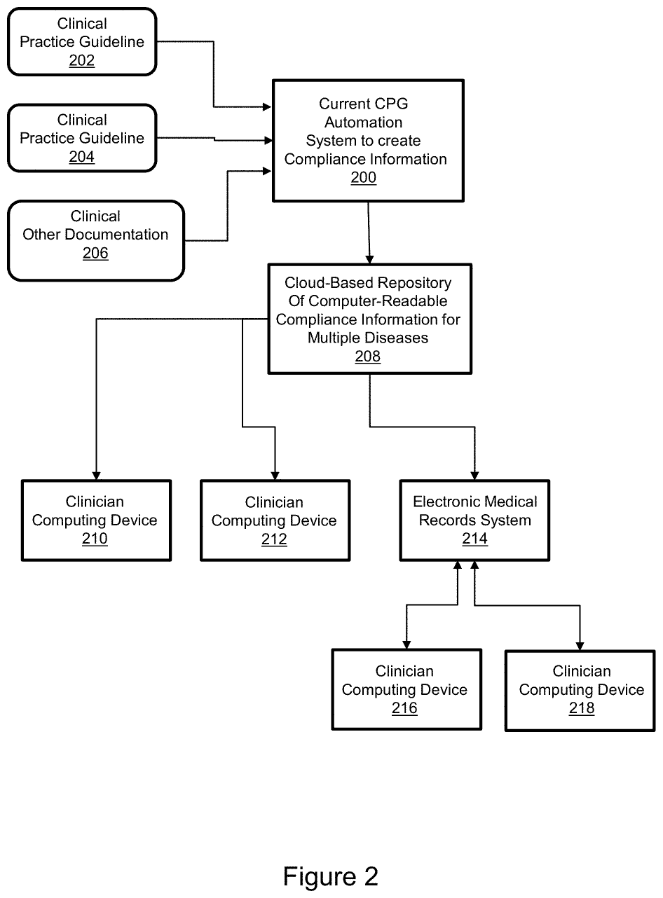

FIG. 2 illustrates a system in which the method of FIG. 1 is implemented. A current clinical practice automation system 200 receives as input multiple clinical practice guidelines 202, 204, 206. The current CPG automation system may include a non-volatile storage repository for storing the computer-readable Compliance Information and one or more computers such as servers, each with memory, storage, processors, and computer programs for translating the clinical practice guidelines into computer-readable Compliance Information. Compliance Information includes, but may not be limited to: compliance rules for recommendation contained in the best practice guideline, a table of the KBI key elements for which values must be provided to determine that compliance with each of the CPG recommendations has been met, a table showing the relationship between recommendations to ascertain if full compliance has been met and other supporting documents. The computer-readable Compliance Information for multiple diseases are uploaded to a Cloud-based repository 208. The computer-readable Compliance Information and recommendations are distributed from the Cloud-based repository 208 to clinical computing devices 210, 212 and electronic medical records system 214. The electronic medical records system 214 may use Compliance Information to modify its user interface to gather disease-specific data and update records with disease-specific data or exchange data with clinician computing devices 216, 218, which may return information associated with disease-specific data to the electronic medical records system. Clinician computing devices 210, 212 may maintain generated records and/or store the records in a repository.

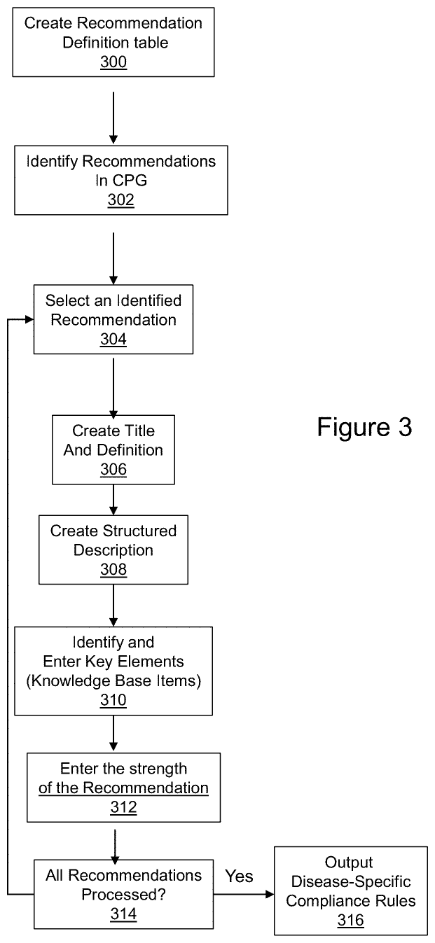

FIG. 3 illustrates a process for defining structured recommendations extracted from a practice guideline. Step 300 is copying the Recommendation Definition template to create a Recommendation Definition table. Step 302 is identifying the recommendations in the disease-specific CPG. Step 304 is selecting a recommendation identified in step 302. The recommendations may be selected in any order based on a wide variety of selection criteria. Step 306 is creating a title and definition for the selected recommendation. Step 308 is creating a structured description for the selected recommendation. Step 310 is identifying and entering the KBI key elements from the structured description into the Recommendation Definition table. Step 312 is entering the strength of the recommendation. Examples of strength values may include Optional, Recommended, and Strongly-recommended. Steps 304 through 312 are iterated until all recommendations have been processed as determined in step 314, following which the completed Recommendation Definition table is outputted in step 316.

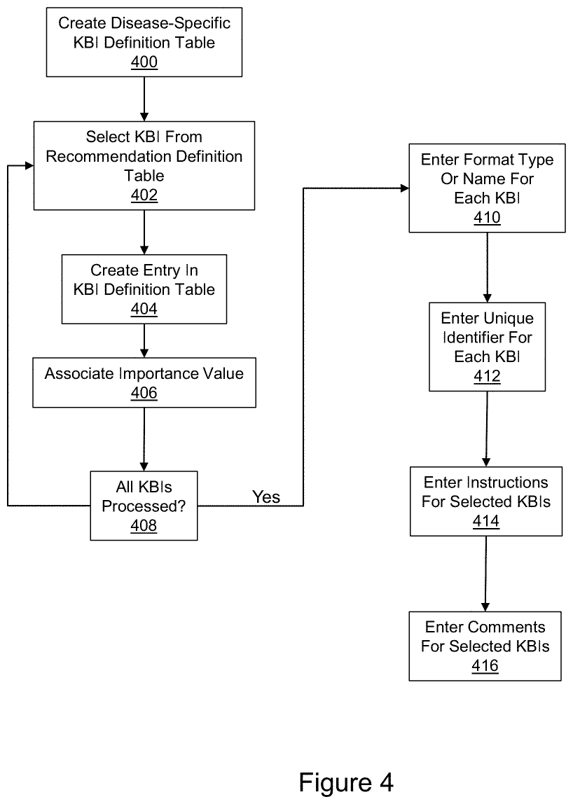

FIG. 4 illustrates a process for defining the KBI key elements associated with the Recommendation Definitions. Step 400 is creating a disease-specific KBI Definition table. Step 402 is selecting a KBI from the recommendation definition table. Step 404 is creating and entering the selected KBI in the KBI Definition table. Step 406 is entering the strength value of the recommendation that is the source of the KBI. Steps 402 through 406 are iterated until all KBIs have been processed as determined in step 408, after which a format type or value-list name is entered for each KBI as indicated in step 410. Step 412 is entering a unique identifier for each KBI. Step 414 is entering instructions for selected KBIs. Step 416 is entering comments for selected KBIs. Instructions and comments are optional, and inclusion may be determined based on a wide variety of factors.

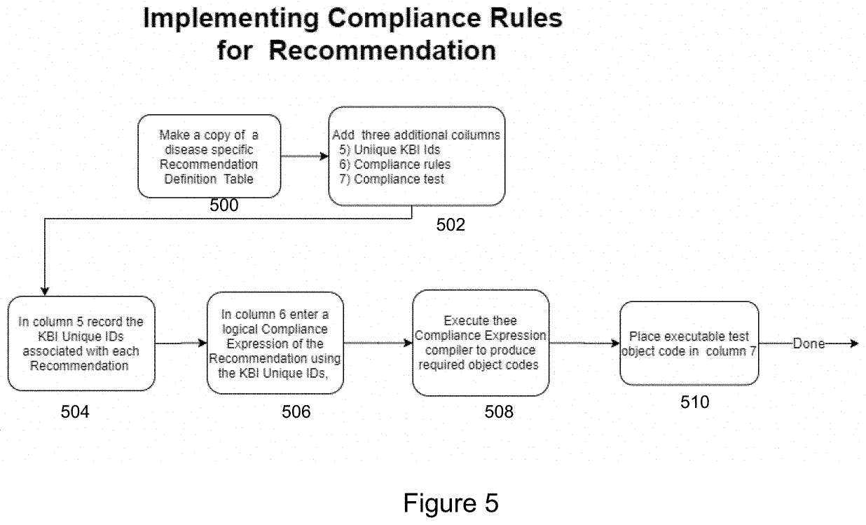

FIG. 5 illustrates implementation of Compliance Rules for recommendations associated with a specific disease. The implementation is described with reference to a Recommendation Definition table including rows and columns. Step 500 is making a copy of a disease-specific Recommendation Definition table. Step 502 is adding 3 columns, column 5 for unique KBI IDs, column 6 for compliance rules, and column 7 for compliance test. Step 504 is inserting the set of KBI Unique IDs associated with a recommendation. Step 506 is entering a logical computer-readable Compliance Rule expression for the recommendations, using the KBI unique IDs associated with the recommendation. Step 508 is executing the Compliance Rule expression compiler to generate object codes, including executable test object code. Step 510 is placing the executable test object code in column 7.

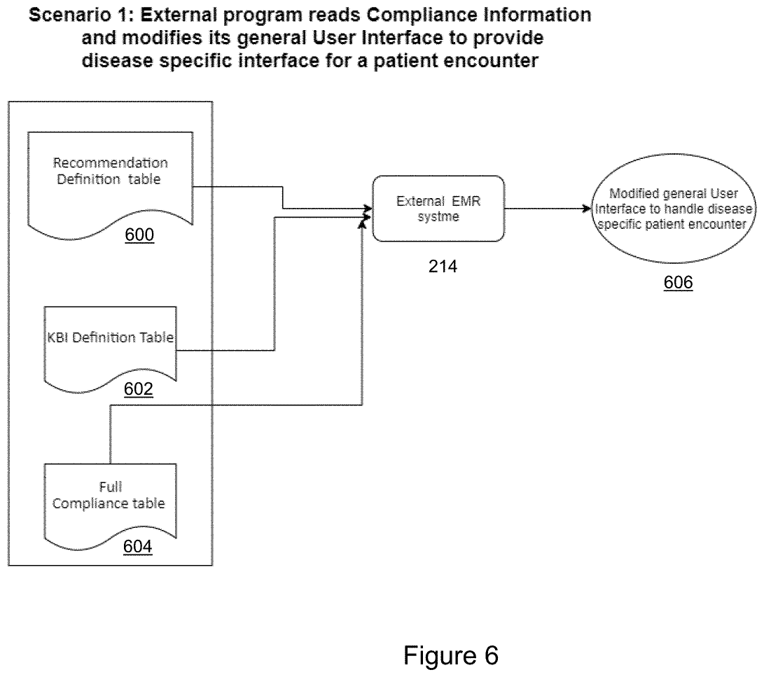

FIG. 6 illustrates automatic creation of a disease-specific UI on the clinician computing device in a scenario in which an external program reads Compliance Information and modifies a general UI to cover a disease-specific patient encounter. Outputs of the Recommendation Definition table 600, the KBI Definition table 602, and Full Compliance table 604 are provided as inputs to the external EMR system 214. The EMR system generates a modified general user interface 606 that is customized for disease-specific patient encounters.

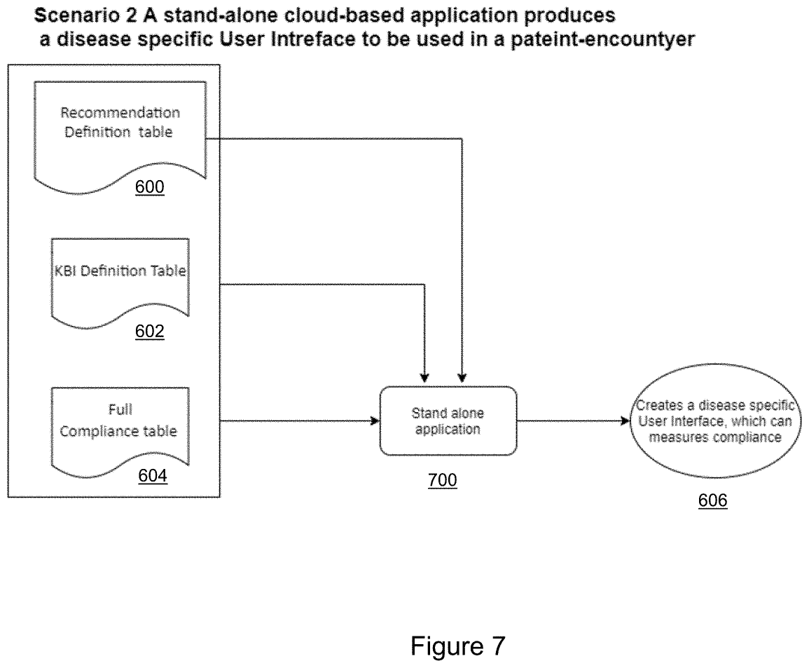

FIG. 7 illustrates automatic creation of a disease-specific UI on the clinician computing device in a scenario in which a standalone application or a Cloud-based application generates the disease-specific UI. Outputs of the Recommendation Definition table 600, the KBI Definition table 602, and Full Compliance table 604 are provided as inputs to a standalone or Cloud application 700. The application generates a UI 606 that is customized for disease-specific patient encounters.

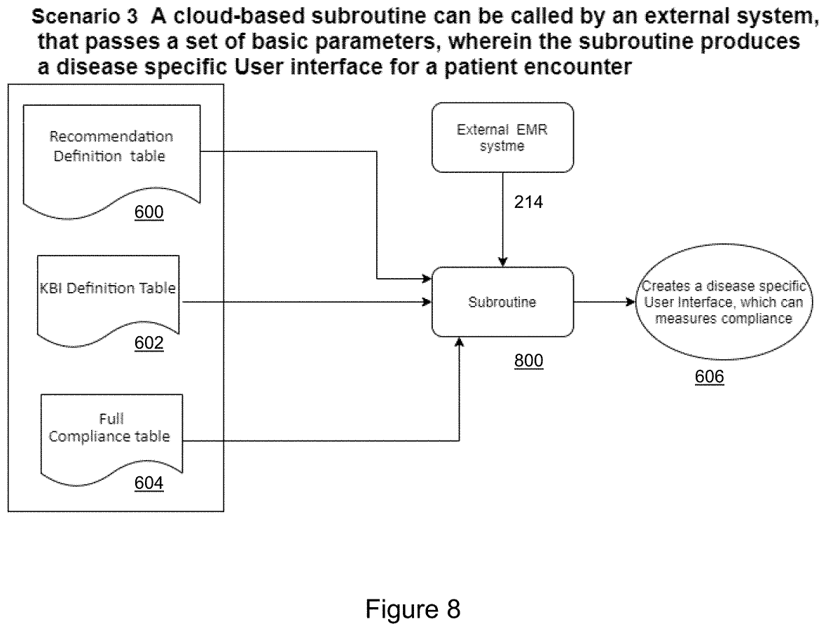

FIG. 8 illustrates automatic creation of a disease-specific UI on the clinician computing device in a scenario in which a Cloud-based subroutine is called by an external program. Outputs of the Recommendation Definition table 600, the KBI Definition table 602, and Full Compliance table 604 are provided as inputs to a subroutine 800. The subroutine is called by the EMR system 214 and generates a modified general user interface 606 that is customized for disease-specific patient encounters.

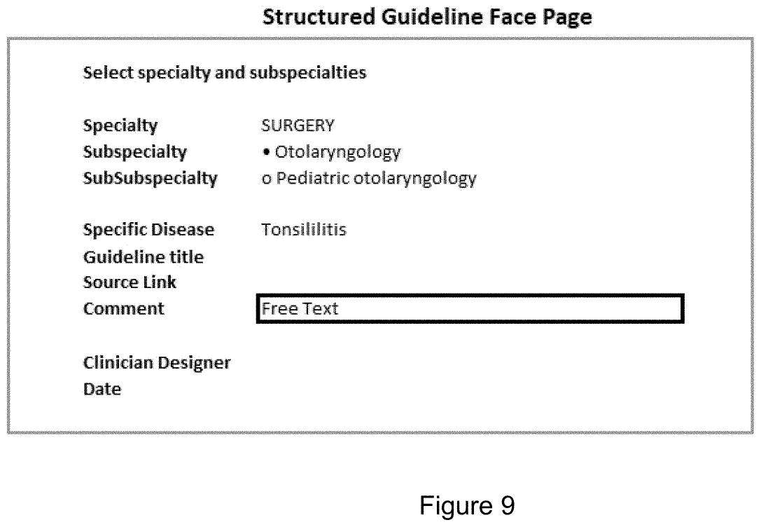

FIG. 9 illustrates a table that identifies the disease to be represented by the Compliance Information, the title of the textual Clinical Practice Guideline that is to be structured, and a link to that textual CPG.

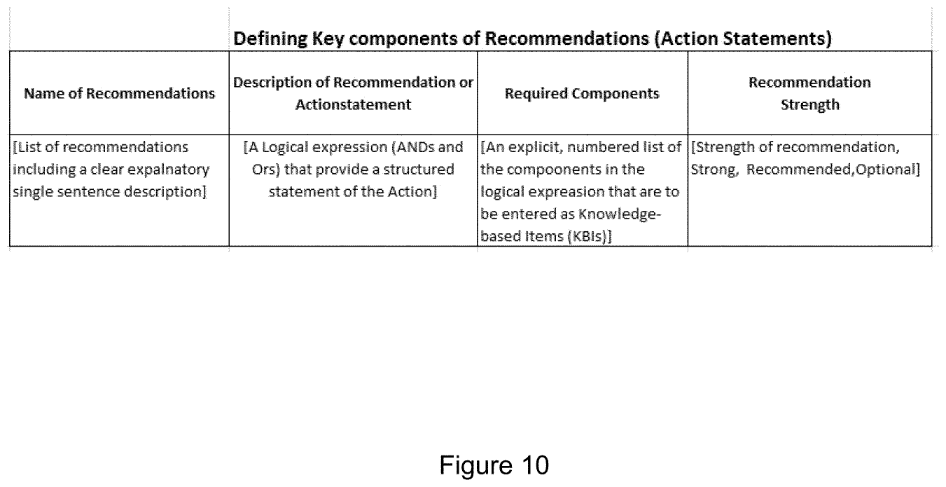

FIG. 10 illustrates a template for a Recommendation Definition table that defines the structured components of a recommendations in a disease-specific CPG. For each recommendation in a resulting table, Column 1 will contain a recommendation label, e.g., “Action Statement 1”, plus a sentence that describes the recommendation; Column 2 will contain a structured description of the recommendation, the description may be an expression that identifies the KBI key elements in the recommendation, connecting KBIs with the logical operators, AND, OR, XOR. For example, if a recommendation in the CPG is “For a diagnosis of tonsillitis prescribe penicillin”, the KBIs would be “diagnosis tonsillitis” and “prescribe penicillin” and the logical expression would be “diagnosis tonsillitis AND prescribe penicillin”; Column 3 will contain a list of the KBI key elements KBIs are considered fundamental entities in the vocabulary of patient-encounters which may appear in many recommendations across multiple CPG. Column 4 will contain a keyword identifying the strength of the recommendation, e.g., Strongly recommended, Recommended, Optional.

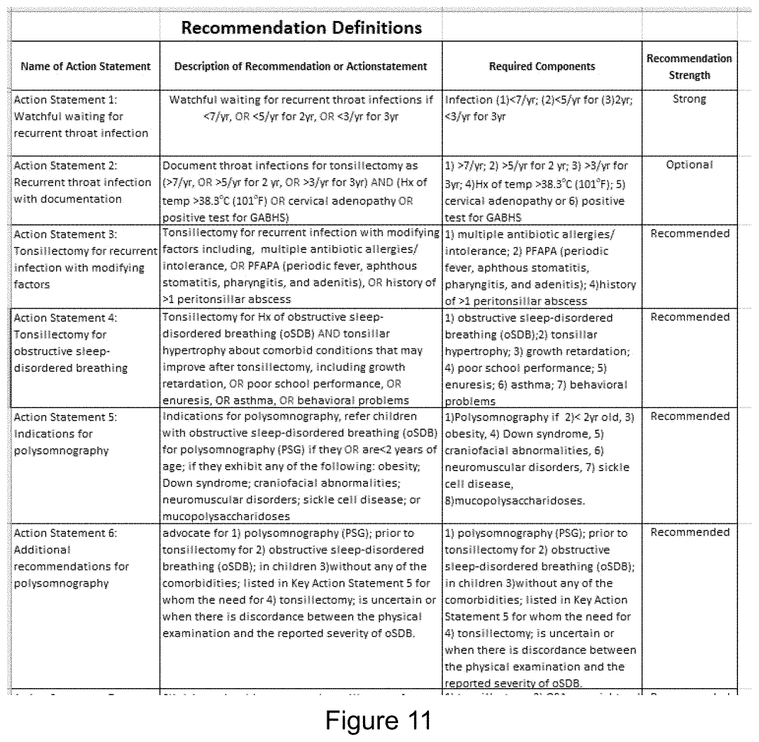

FIG. 11 illustrates a table of structured Recommendation Definitions derived from a CPG for pediatric tonsillitis. These Recommendations were extracted from the CPG produced by the Otolaryngology Academy. Their Guidelines refer to Recommendations as Action Statements. FIG. 11 covers the first half of the pediatric tonsillitis Recommendations.

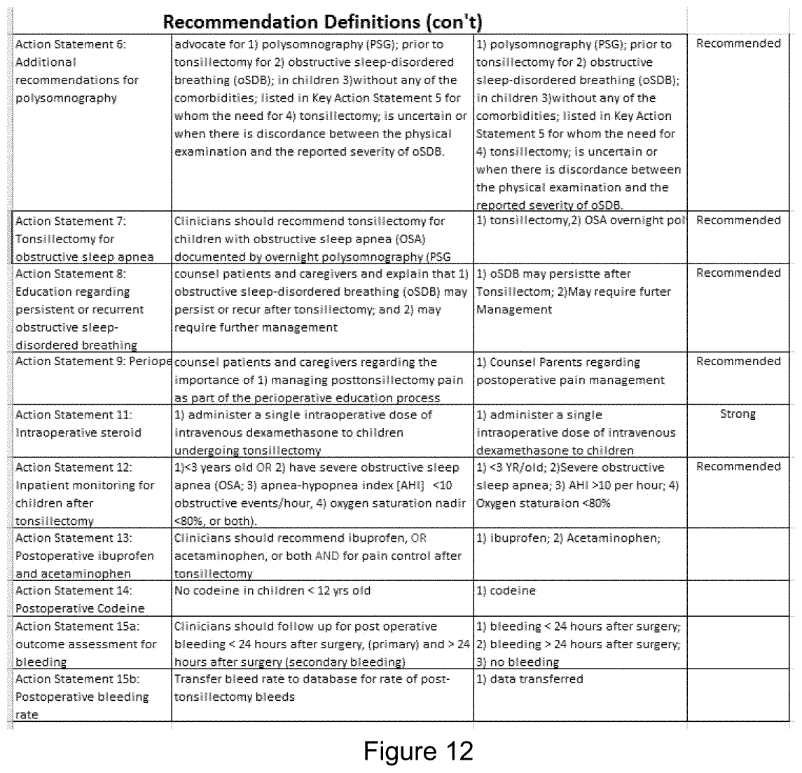

FIG. 12 illustrates the second half of the Recommendation Definitions table for pediatric tonsillitis.

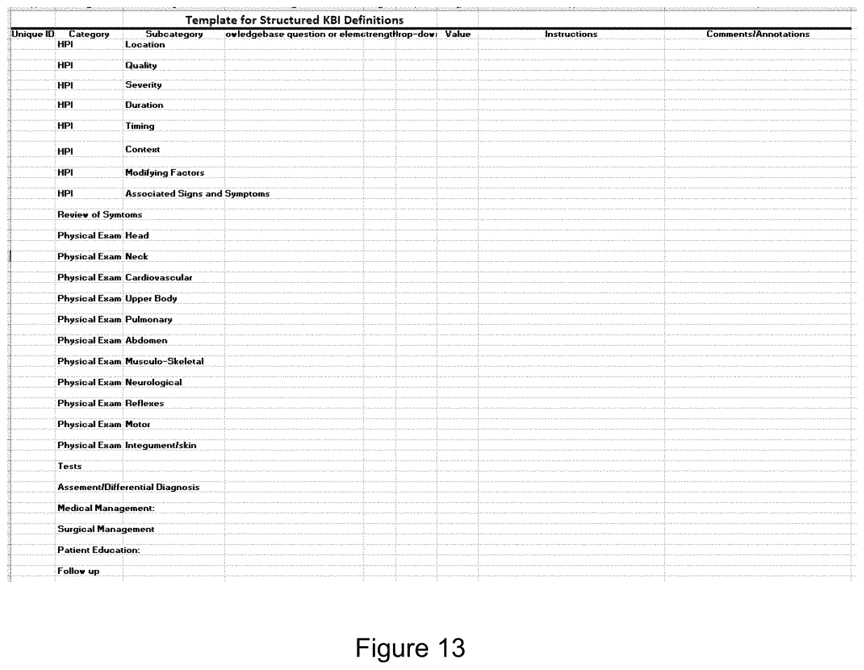

FIG. 13 illustrates a template for a KBI Definition table. The table produced using this template will define the components of the KBIs that were uncovered by structuring the Recommendations (see FIGS. 10 and 11). The template is organized so that KBIs can be entered into categories that coincide with the standard procedure that Clinicians generally follow during a patient-encounter. This procedure is usually followed no matter what specialty the Clinician practices. For example, the KBI “Diagnosis Tonsillitis” would be placed in the Assessment/Differential Diagnosis category and “Prescribe penicillin” would be placed in either the medical management or Surgical management categories.

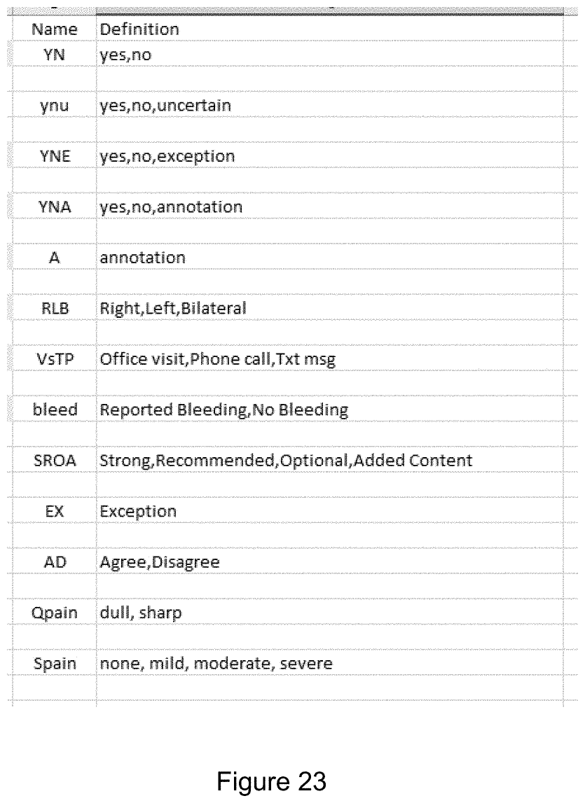

The categories as presented in the template are as follow:
Category 1 HPI (History of Present Illness)—HPI includes KBIs that deal with the history of the illness presented by the patient. The HPI category has subcategories that include Location of the symptoms, the Quality of the symptoms, the Severity of the symptoms, the Duration or the length of time that symptoms have been present, the Timing or how often the symptoms have occurred, the Context of the symptoms such as the age of the patient, any Modifying Factors that may affect the symptoms, diagnosis or treatment, and Signs and Symptoms that may be associated with the primary symptoms.
Category 2 Review of Symptoms—Review of symptoms is a mandated summary of the general condition of the patient. There are no disease-specific KBIs associated with this category.
Category 3 Physical Exam—These are KBIs associated with a physical exam of the patient (there are numerous sub-categories covering different physical aspects of the patient). In a table for a specific disease only those aspects related to the symptoms will appear, e.g., for tonsillitis, entries may only be entered for Head and neck.
Category 4 Test—These are KBIs for tests that are related to the Recommendations Category 5 Assessments/Differential Diagnosis—These are KBIs that are associated with the conclusion of the examination of the patient. They include the diagnoses categories associated with the symptoms as well as assessments of key related conditions.
Category 6 Medical Management—These are KBIs that relate to medical management and treatment of the diagnosed disease.
Category 7 Surgical Management—These are KBIs that relate to treatment that involves surgery. Surgical Management will only be an option for a certain diagnosis.
Category 7 Patient Education—These are KBIs that involve informing and educating the patient about the diagnosis and treatment, i.e., what the patient should expect and what the patient or caregiver should do.
Category 8 Follow Up—These are KBIs for actions or activities that should be followed up after the initial treatment.
The components of each KBI are to be entered in the following columns:
Column 1 contains a unique ID that must be created for every KBI entered into the KBI Definition table. This provides a computer-readable reference to that KBI. In particular, Compliance Rules will utilize these unique IDs.
Column 2 contains the Category for which the KBI belongs.
Column 3 contains the subcategory for which the KBI belongs if that is appropriate, otherwise the entry will be blank.
Column 4 contains a mathematical expression that defines the KBI. This expression may be extracted from the logical expression of its associated recommendation.
Column 6 contains the strength of the Recommendation from which the KBI was identified. In many cases the values allowed in this column will be: Strongly Recommended, Recommended, Optional.
Column 7 will contain an indication of the value type to be entered for a KBI when it is presented in a generated patient-encounter User Interface. Allowed entries in this column include standard datatypes, e.g., “text”, “integer”. In addition, the column entry can be a name of a “value-list” of allowed values (see FIG. 23). For example, a value-list name could be “YN”, which could identify a list with [“yes” “no”] as allowed values, or a value-list name “YNU”, which could identify a list with [“yes” “no” “uncertain”] as allowed values. An example of a value-list can be seen in FIG. 23. KBI may be architected to accept a value-list as opposed to a simple data format so that value entries in the patient-encounter User Interface can be well defined.
Column 8 contains entered test values for a KBI. The invention offers a test facility so that as Recommendations and KBIs are defined, they can be tested to make sure they are properly structured.
Column 9 optionally contains instructions to be incorporated into the generated patient-encounter User Interface. If no instructions are warranted the column will remain blank.
Column 10 contains any comment that may elucidate the KBI further. If no comment is warranted the column remains blank.

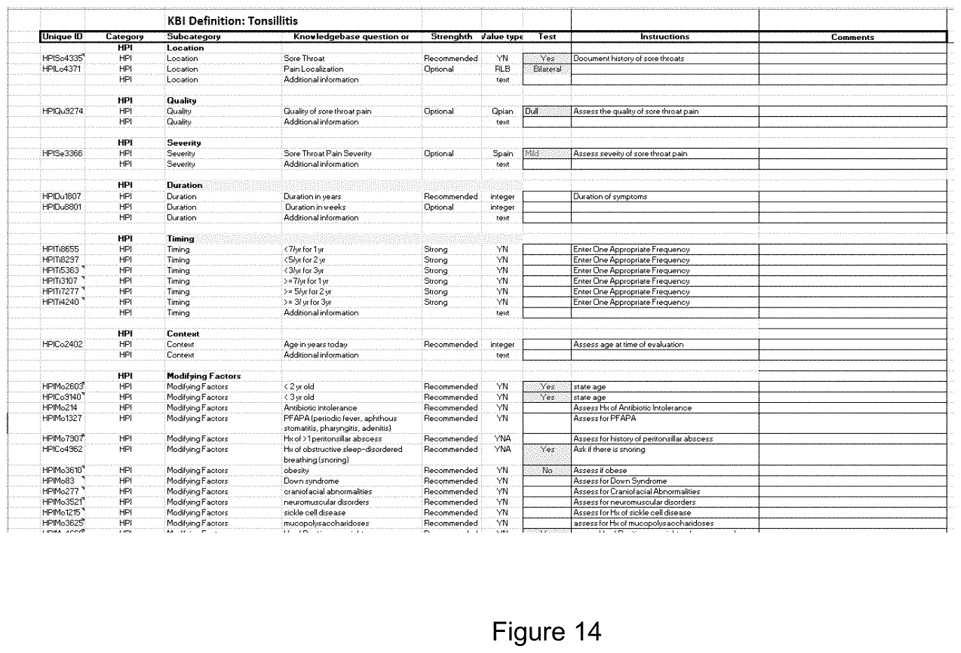

FIG. 14 illustrates, as an example, the first section of a KBI Definition table for pediatric tonsillitis.

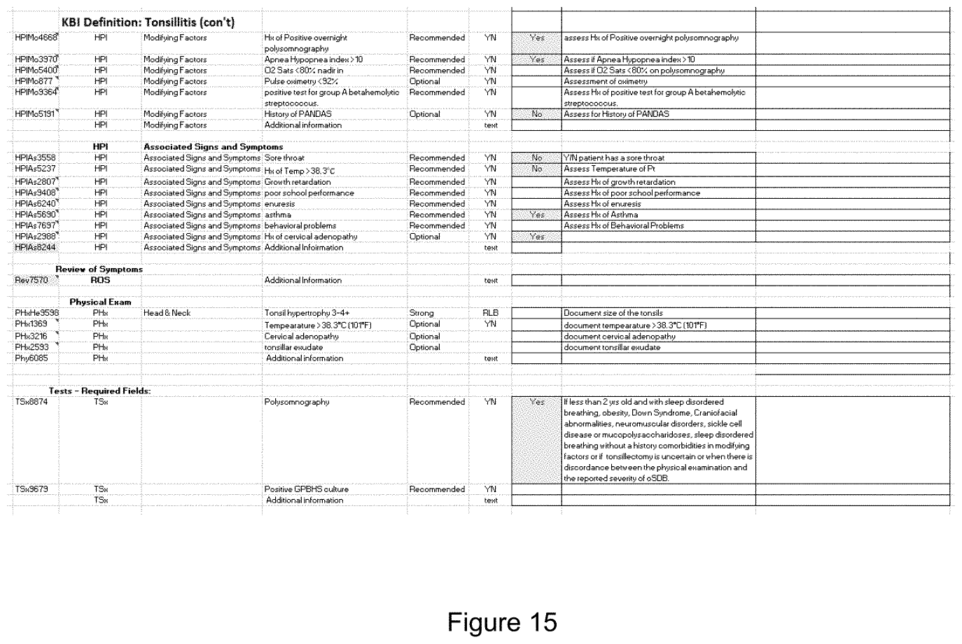

FIG. 15 illustrates the next section of the KBI Definition table for pediatric tonsillitis.

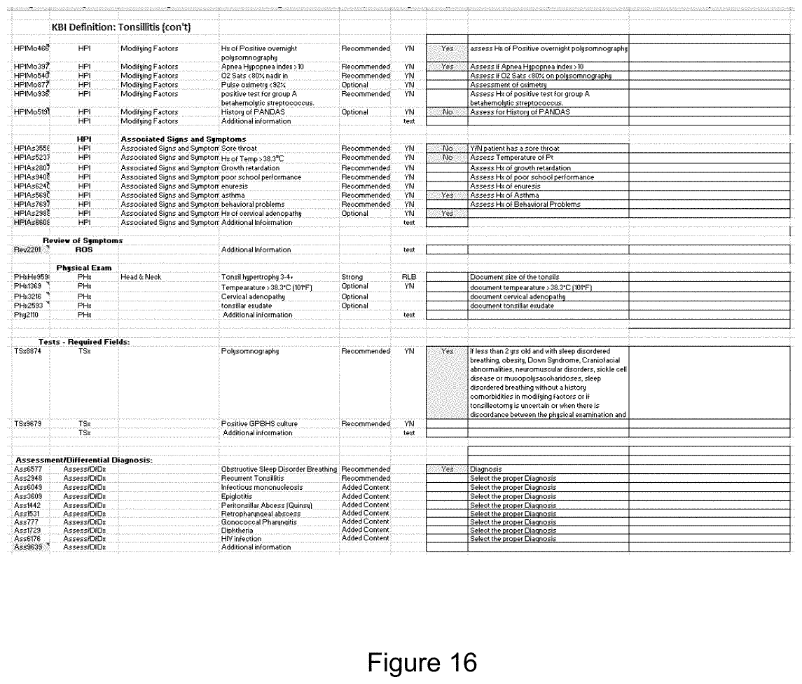

FIG. 16 illustrates the next section of the KBI Definition table for pediatric tonsillitis.

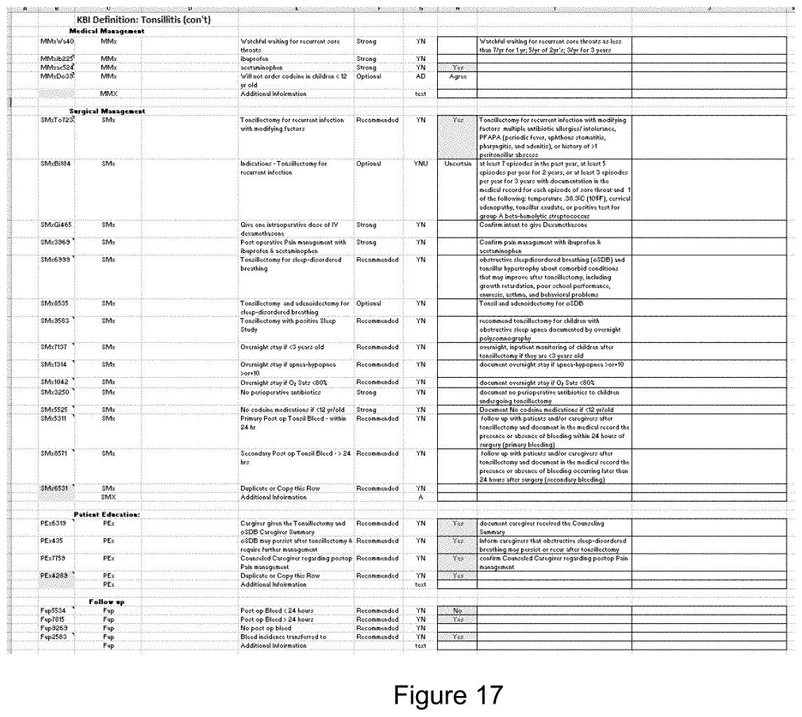

FIG. 17 illustrates the next section of the KBI Definition table for pediatric tonsillitis.

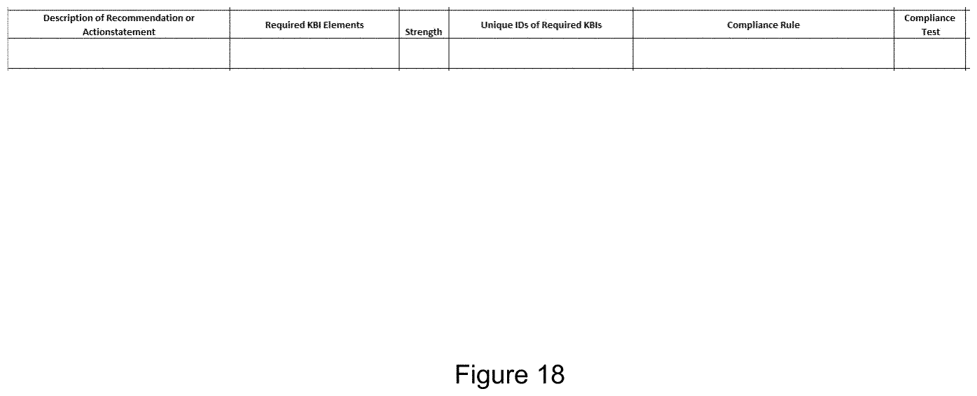

FIG. 18 illustrates a template of a table used to define the machine-readable Compliance Rules for the set of Recommendations. The first four columns of the table that is defined by the template are a direct copy of the Recommendation Definition table. These columns are included in this table for reference to assist in the authoring of machine-readable Compliance Rules expressions. Three additional columns are defined as column 5 through column 7.

Column 5 contains the unique IDs of the set of KBIs associated with a recommendation that were originally extracted from each recommendation in the Recommendation Definition table and entered into the KBI Definition table.
Column 6 contains a logical statement, based on a well-defined syntax, to describe the Compliance Rule for each Recommendation. The syntax includes standard logical operator of AND, OR, and XOR as well as the binary operator of =,< >, >, <. The operands of these operators are the KBI Unique IDs or constants. The syntax allows the standard use of parentheses if an operand is an expression that is more complicated than a single Unique ID. Standard arithmetic operators are allowed as part of an expression. The syntax also allows for internal commenting by surrounding the comment with brackets ([ ]). An example of a Compliance Rule written in this syntax is: IF(HPITi8655 [<7/yr” ] OR HPITi8655 [<5/yr for 2 yr] OR HPITi5363 [3/yr for 3 yr]=“yes” The example illustrates one use of the commenting capability, which is to include the KBI description associated with the Unique ID to make the Compliance Rule more human readable.
Evaluating the Compliance Rule is the primary measure of compliance for a recommendation. If a Compliance Rule is evaluated as True, using the values entered for the KBI in Patient-Encounter User Interface, then the Clinician will have complied with the recommendation. If the Compliance Rule is evaluated as not True, then the Clinician will not have complied with the recommendation, unless the Clinician has entered an annotation to explain why a compliant value for the KBI was not entered.
Column 7 supports a capability for testing whether the Compliance Rule is properly constructed. If values are entered into the test column (Column 8 of the KBI Definition table) then this column will indicate whether a recommendation is compliant. (This column is not shown in the Figure to allow for a larger image of the other columns.)

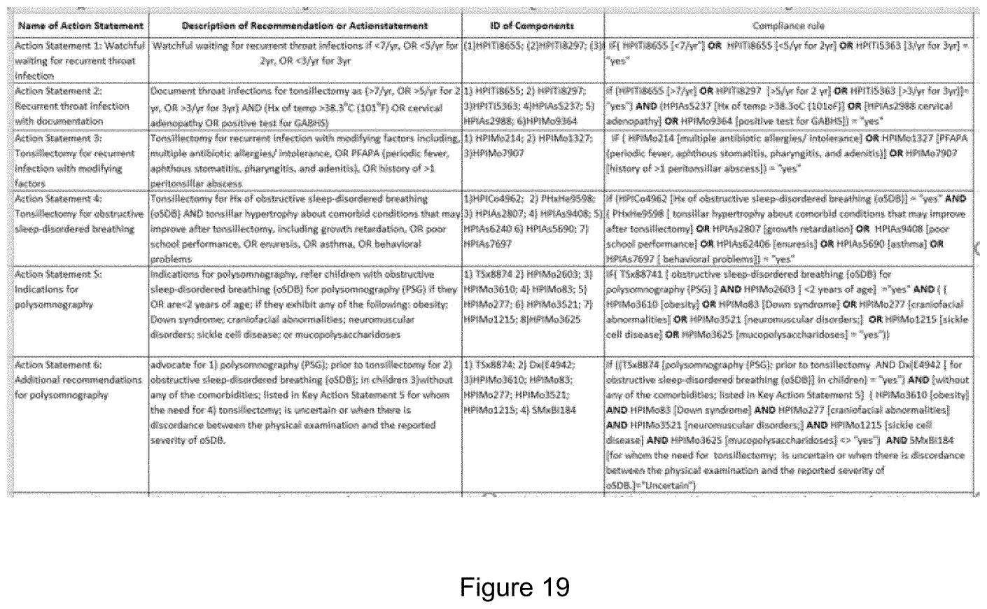

FIG. 19 illustrates the first section of a table of the Compliance Rules for recommendations for pediatric tonsillitis.

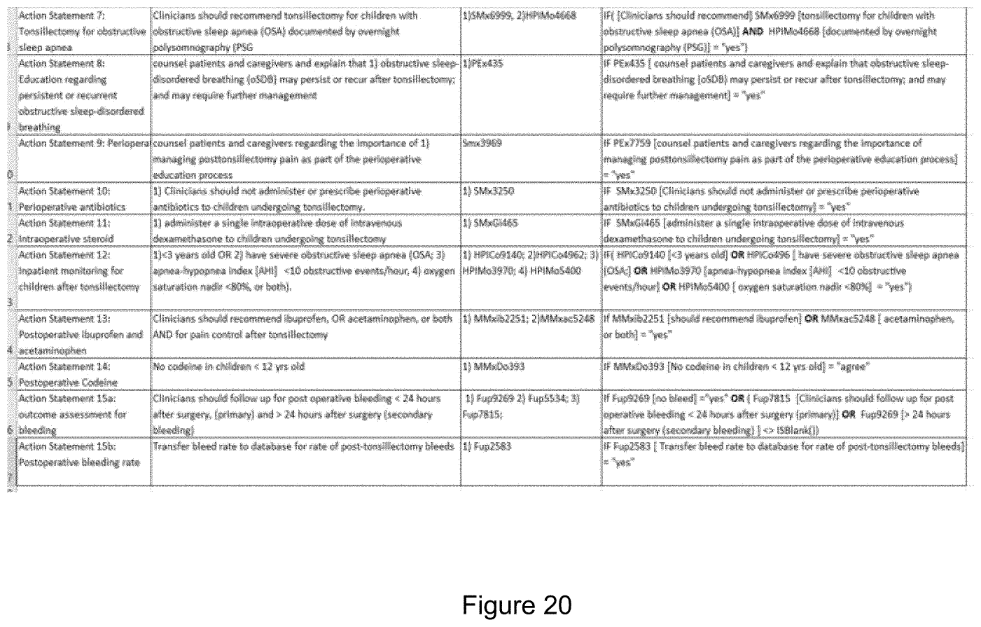

FIG. 20 illustrates the second section of a table of the Compliance Rules for recommendations for pediatric tonsillitis.

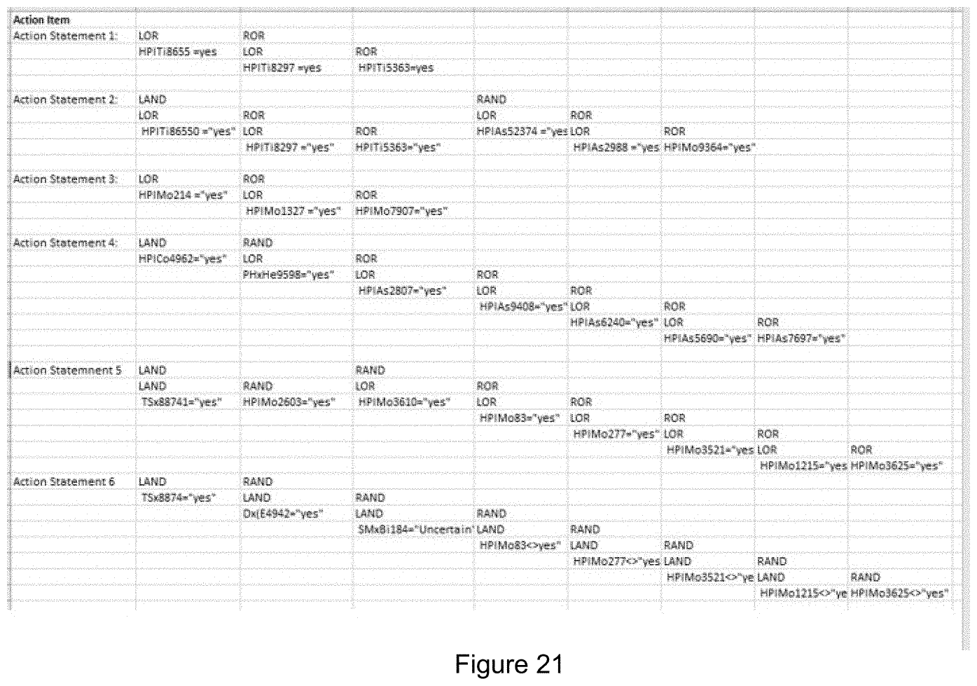

FIG. 21 illustrates machine readable object data structure of compiled Compliance Rules for pediatric tonsillitis. One embodiment of the invention will include a complier that can compile the Compliance Rules to produce an object data structure that will enable external programs to integrate the Compliance Rules without needing their own interpreter for the Rules. The compiler will be able to produce various forms of an object data structure. This figure displays an object data structure in a table that stores the compiled Compliance Rules as binary trees.

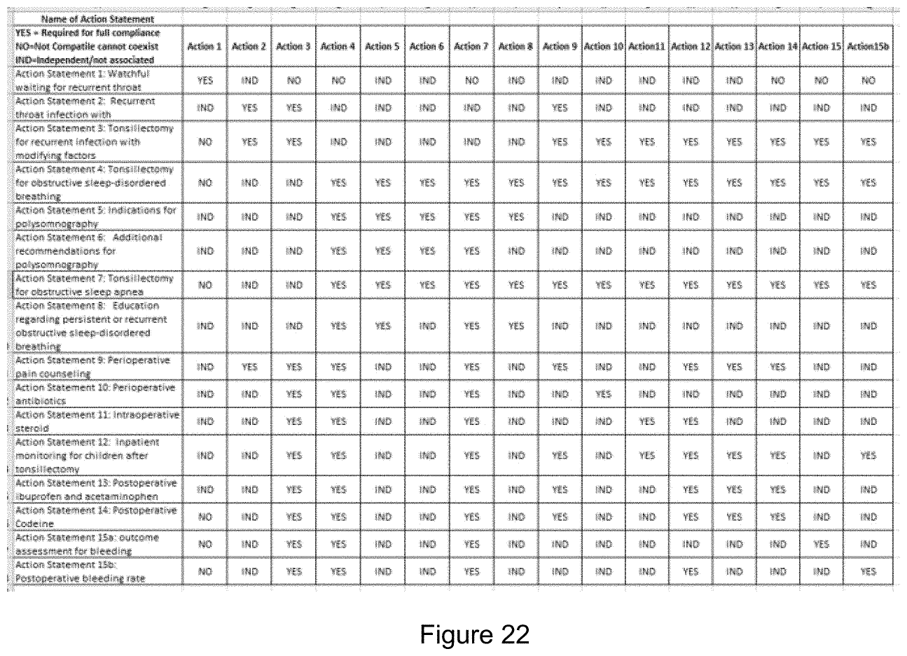

FIG. 22 illustrates a table displaying the relationships among the recommendations for pediatric tonsillitis. To achieve full compliance with a CPG, the relationships between recommendations must be considered. For example, one recommendation for pediatric tonsillitis is for medical management and calls for watchful waiting. Another recommendation is for Surgical management and calls for a tonsillectomy. While values for the KBIs entered into a Patient-Encounter User Interface for both recommendations may independently show compliance, clearly both recommendations are incompatible and cannot exist together. For other recommendations two or more should be compliant for there to be full compliance with the CPG. The table illustrated in FIG. 17 displays the relationship among all recommendations where column entries with the value “YES” for a particular row Recommendation display the other recommendation that should also be compliant to achieve full compliance, where column entries with a “NO” imply incompatibility even though the recommendation associated with that column is compliant and where column entries with a “IND” indicate Recommendations that do not have a relationship with the row-Recommendation.

FIG. 23 illustrates a value list for the key elements for pediatric tonsillitis. Column 1 of the value list provides the name of the list. Column 2 contains the keyword values in the list. The value-list names are entered into column 6 of the KBI Definition table. List of entries are associated with the column 7, the test column in the KBI Definition table. The list is also a component of the Compliance Information, so that the generated UI for a patient encounter can restrict entries to the list.

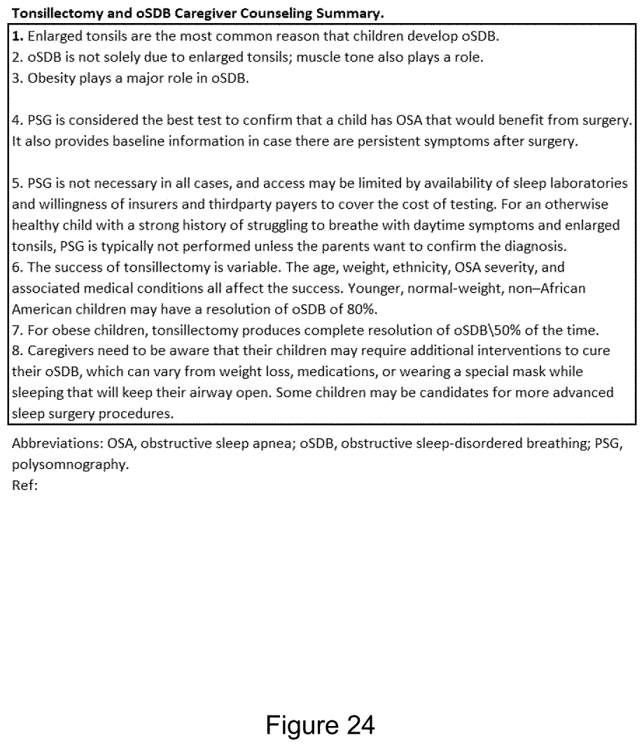

FIG. 24 illustrates Counselling summary for pediatric tonsillitis. Supporting documentation that is associated with a KBI or Recommendation that can be extracted from the CPG is often valuable. This figure displays a counselling summary to support the pediatric tonsillitis KBI that covers patient education for a patient that has had a tonsillectomy and has obstructive sleep disorder breathing.

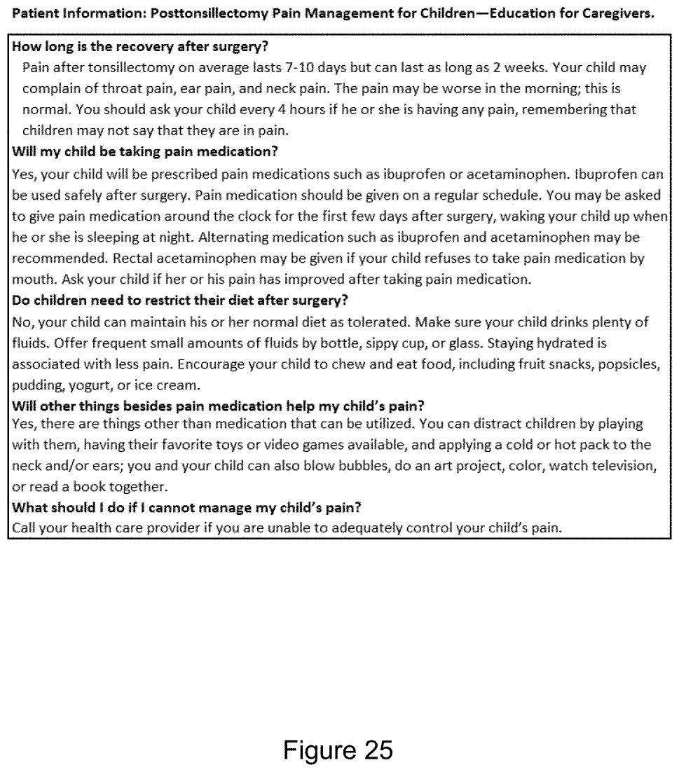

FIG. 25 also illustrates auxiliary information to support a Patient Educations KBI. The information supports pain management education by displaying answers for commonly asked question by a care giver.

FIG. 9 through FIG. 25 display artifacts that are contained in Compliance Information for a specific disease. The Compliance Information can be used to generate a disease-specific Patient-Encounter User Interface with a supporting application. This generated user interface will guide the Clinician to best practice by enabling the disease-specific recommendations from a CPG and providing measurement and documentation as to how well the clinician has been compliant with those recommendations.

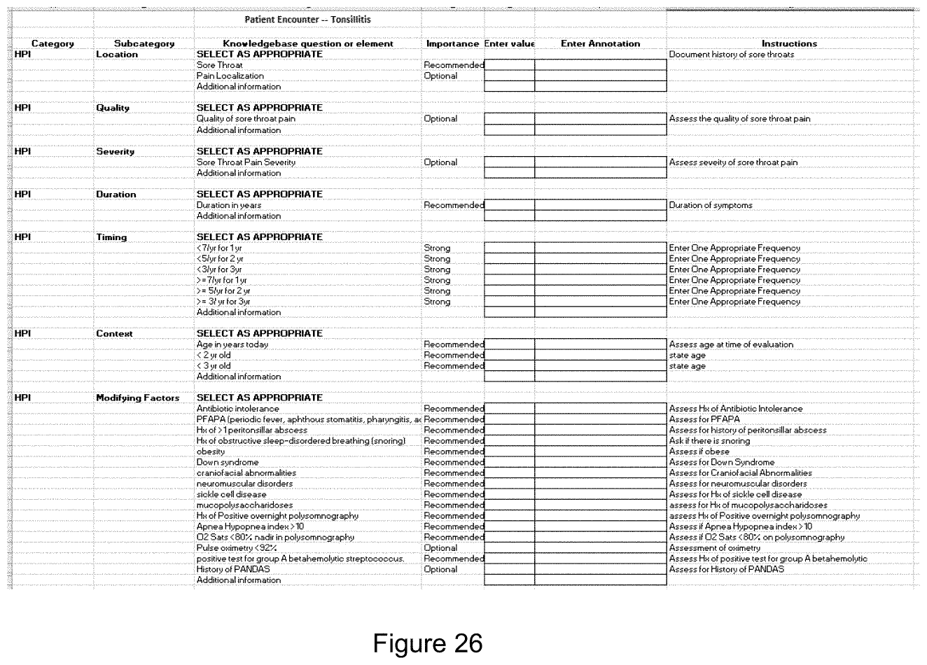

FIG. 26 illustrates the first section of a generated Patient Encounter User Interface for pediatric tonsillitis. The embodiment of the current invention includes a process where Compliance Information is used as input to create a stand-alone application on a computer device or in the Cloud. FIG. 26 displays the User Interface that is generated for pediatric tonsillitis.

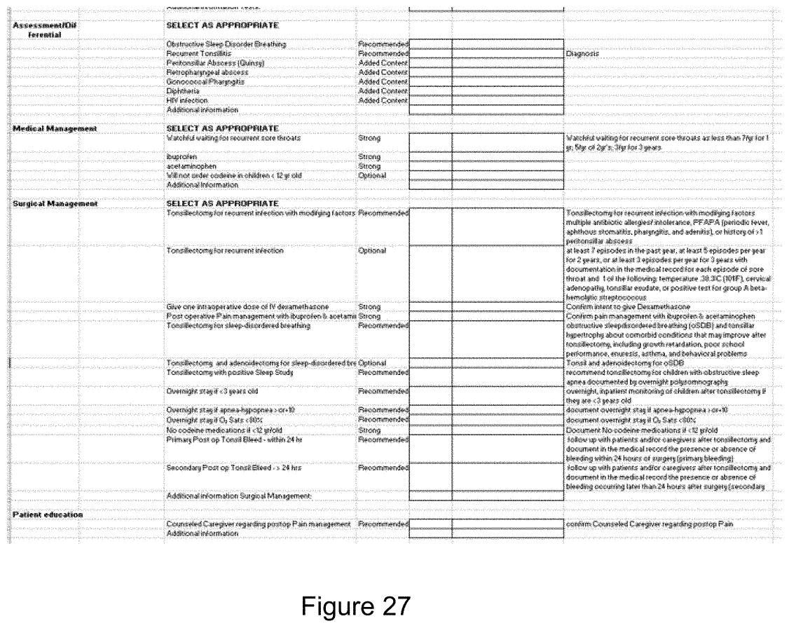

FIG. 27 illustrates the second section of a generated Patient Encounter User Interface for pediatric tonsillitis.

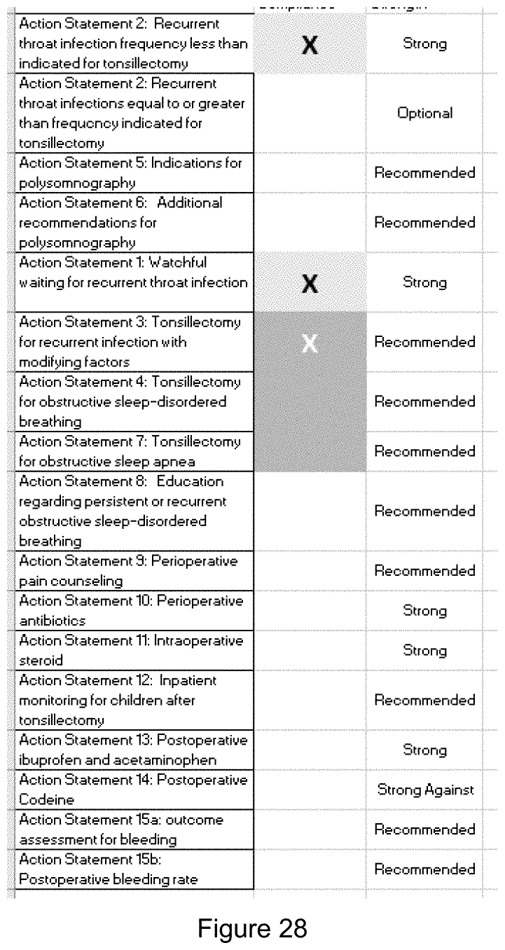

FIG. 28 illustrates a runtime assessment of full compliance and incompatibilities for the values entered into the User Interface during a patient encounter where the preliminary diagnosis was pediatric tonsillitis. For any particular recommendation, the background color green implies that these other recommendations are related and should also be compliant to achieve full compliance. The background color red implies that these other recommendations are incompatible and should not be compliant. The X in the square indicates which recommendations are compliant based on inputed values and their Compliance Rule.

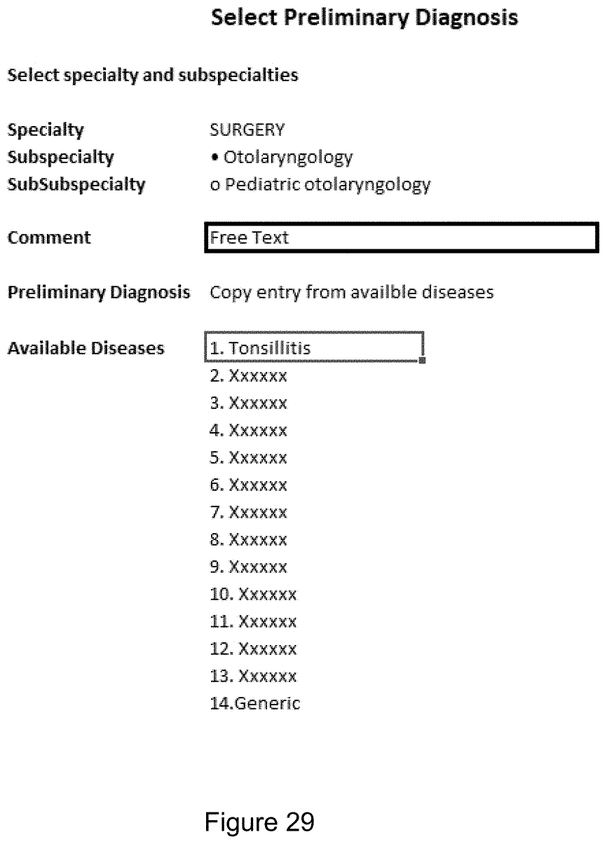

FIG. 29 illustrates a generated face page for a patient encounter UI, where pediatric tonsillitis is chosen as the preliminary diagnosis for pediatric tonsillitis.

A number of features, aspects, embodiments, and implementations have been described. Nevertheless, it will be understood that a wide variety of modifications and combinations may be made without departing from the scope of the inventive concepts described herein. Accordingly, those modifications and combinations are within the scope of the following claims.

What is claimed is:

1. A method for implementing and verifying compliance with current standards of care, comprising:
creating computer-readable Compliance Information that represents recommendations of current clinical practice guidelines associated with a specific disease, wherein the Compliance Information comprises compliance rules for evaluating adherence with the recommendations as a function of values of key elements;
distributing the Compliance Information to each of a plurality of computing devices, each comprising a microprocessor and memory;
contemporaneous with a patient encounter wherein the specific disease is indicated by a preliminary diagnosis, generating a patient-specific record with one of the computing devices by:
generating a user interface on the computing device based on the distributed Compliance Information;
prompting gathering of data needed to assign values to the key elements; evaluating the compliance rules using the values assigned to the key elements;
prompting compliance with recommendations for which compliance is not indicated by evaluated ones of the compliance rules; and
generating a verification of compliance with the current standards of care in response to compliance with the recommendations as indicated by evaluated ones of the compliance rules.

2. The method of claim 1 wherein creating the computer-readable Compliance Information that represents the recommendations of current clinical practice guidelines associated with the specific disease comprises representing the compliance rules as logic statements.

3. The method of claim 2 wherein creating the computer-readable Compliance Information that represents the recommendations of current clinical practice guidelines associated with the specific disease comprises creating the key elements as variables of the logic statements.

4. The method of claim 1 wherein creating the computer-readable Compliance Information that represents the recommendations of current clinical practice guidelines associated with the specific disease comprises including only recommended and strongly recommended recommendations and action statements of the current clinical practice guidelines.

5. The method of claim 1 wherein creating the computer-readable Compliance Information that represents the recommendations comprises determining a minimal set of key elements that are required to assure compliance with the recommendations.

6. The method of claim 1 wherein prompting gathering of the data needed to assign values to the key elements comprises designating as inapplicable at least one key element determined to be unnecessary based on the data obtained during the patient encounter.

7. The method of claim 1 wherein generating the verification of compliance with the current standards of care in response to compliance with the recommendations as indicated by evaluated compliance rules comprises designating as inapplicable at least one recommendation determined to be unnecessary based on the data obtained during the patient encounter.

8. The method of claim 1 comprising recording an annotation specific to the patient-encounter and associating the annotation with one of the key elements.

9. The method of claim 1 wherein distributing the Compliance Information to each of the computing devices comprises distributing Compliance Information associated with a plurality of diseases.

10. The method of claim 1 further comprising creating revised computer-readable Compliance Information that represents revised recommendations of current clinical practice guidelines associated with the specific disease and automatically distributing the revised Compliance Information to each of the computing devices.

11. A compliance automation system for implementing and verifying compliance with current standards of care, comprising:
at least one compute node that generates computer-readable Compliance Information that represents recommendations of current clinical practice guidelines associated with a specific disease, wherein the Compliance Information comprises compliance rules for evaluating adherence with the recommendations as a function of values of key elements;
a non-volatile storage repository via which the Compliance Information is distributed to each of a plurality of clinician computing devices, each comprising a microprocessor and memory; and
program code that runs on one of the clinician computing devices contemporaneous with a patient encounter for which the specific disease is indicated by a preliminary diagnosis, the program code comprising instructions that:
generate a user interface on the clinician computing device based on the distributed Compliance Information;
prompt gathering of data needed to assign values to the key elements; evaluate the compliance rules using the values assigned to the key elements;
prompt compliance with recommendations for which compliance is not indicated by evaluated ones of the compliance rules; and
generate a verification of compliance with the current standards of care in response to compliance with the recommendations as indicated by evaluated ones of the compliance rules.

12. The compliance automation system of claim 11 wherein the compliance rules comprise logic statements.

13. The compliance automation system of claim 12 wherein the key elements comprise variables of the logic statements.

14. The compliance automation system of claim 11 wherein the computer-readable Compliance Information that represents the recommendations of current clinical practice guidelines associated with the specific disease comprises only recommended and strongly recommended recommendations and action statements of the current clinical practice guidelines.

15. The compliance automation system of claim 11 wherein the computer-readable Compliance Information that represents the recommendations includes only a minimal set of key elements that are required to assure compliance with the recommendations.

16. The compliance automation system of claim 11 wherein the instructions designate as inapplicable at least one key element determined to be unnecessary based on the data obtained during the patient encounter.

17. The compliance automation system of claim 11 wherein the instructions designate as inapplicable at least one recommendation determined to be unnecessary based on the data obtained during the patient encounter.

18. The compliance automation system of claim 11 wherein the instructions record an annotation specific to the patient-encounter and associate the annotation with one of the key elements.

19. The compliance automation system of claim 11 wherein Compliance Information associated with a plurality of diseases is maintained in the repository.

20. The compliance automation system of claim 11 wherein the compute node generates revised computer-readable Compliance Information that represent revised recommendations of current clinical practice guidelines associated with the specific disease and the revised Compliance Information is automatically distributed from the repository to each of the clinician computing devices.
